(19) United States
(12) Patent Application Publication
(10) Pub. No.: US 2013/0289543 A1
(43) Pub. Date: Oct. 31, 2013

(54) SYSTEM AND METHOD FOR IN SITU CREATION OF A SMALL APERTURE INTRAOCULAR LENS

(71) Applicant: David Haydn Mordaunt, Los Gatos, CA (US)

(72) Inventor: David Haydn Mordaunt, Los Gatos, CA (US)

(21) Appl. No.: 13/795,527

(22) Filed: Mar. 12, 2013

Related U.S. Application Data
(60) Provisional application No. 61/636,837, filed on Apr. 23, 2012.

(51) Int. Cl. A61F 9/007

(52) U.S. Cl. CPC A61F 9/00736
USPC 606/5

(57) ABSTRACT

A system and method are provided for altering the optical characteristics of an Intraocular Lens (IOL), in situ, using laser techniques. Specifically, a computer-controlled laser unit either creates microbubbles, or converts inclusions, inside the IOL, to establish a predetermined optical barrier having a predetermined opacity. The resultant optical barrier is oriented in the IOL to control light passing through the IOL, and to thereby minimize or correct adverse optical effects that would otherwise be present.

This application claims the benefit of U.S. Provisional Patent Application Ser. No. 61/636,837, titled SYSTEM AND METHOD FOR IN SITU CREATION OF A SMALL APERTURE INTRAOCULAR LENS, filed Apr. 23, 2012. The entire contents of Application Ser. No. 61/636,837 are hereby incorporated by reference herein.

### FIELD OF THE INVENTION

The present invention pertains generally to systems and methods for altering the optical characteristics of an Intraocular Lens (IOL). More particularly, the present invention pertains to systems and methods for altering the optical characteristics of an IOL, in situ, using laser technology. The present invention is particularly, but not exclusively, useful for systems and methods that alter the optical characteristics of an IOL by creating optical barriers inside the IOL to correct vision defects of a patient.

### BACKGROUND OF THE INVENTION

In the field of optics innumerable systems, devices and methods have been developed and devised to direct and control the effects of light. Of particular importance here is how light can be properly directed and controlled within a transparent material, as the light passes through the material. More specifically, the concern here is for how an Intraocular Lens (IOL) can be altered, in situ, to improve the optical response of the IOL after a cataract procedure. Of particular interest is how this can be done using laser technology.

Typically, an IOL is made of a substantially transparent material such as silicon or modified acrylic. Both of these material types are susceptible to alteration by a laser, and in each instance the optical properties of the material are changed at the site of the alteration. A very useful aspect of this fact is that a laser is capable of altering a material by creating microbubbles that are only a few microns in diameter (e.g. <10 μm). Moreover, by varying the density of these microbubbles, the resultant opacity of the material is likewise varied.

Apart from the creation of microbubbles, it is also known that inclusions can be embedded into a transparent material, and then subsequently converted by an interaction of the inclusion with a laser beam to introduce an opacity into the transparent material. Like the density of microbubbles, the density of converted inclusions will have a direct effect on the level of opacity that is created within the transparent material.

With a view toward using laser alterations in an IOL to improve its optical response, at least two different phenomena are particularly noteworthy. The first involves the so-called “Pinhole Effect” which effectively increases the depth of focus by decreasing the aperture through which light passes. The other involves dysphotopsia, which occurs when light is reflected from boundaries inside an IOL. It is known that this reflected light can cause the sensation of halos around lights at night, or dark regions in the field of vision during the day. In each instance, it is envisioned that the in situ creation of predetermined optical barriers inside an IOL can improve the vision qualities of the IOL.

With the above in mind, it is an object of the present invention to provide systems and methods for selectively creating optical barrier within an Intraocular Lens. Another object of the present invention is to provide systems and methods for using laser systems to effectively alter the optical characteristics of a transparent material to correct a vision defect of a patient. Yet another object of the present invention is to provide systems and methods for the in situ laser alteration of an IOL for correction of a vision defect that is easy to use and is comparatively cost effective.

### SUMMARY OF THE INVENTION

In accordance with the present invention, an IOL is photo-altered, in situ, to correct a vision defect that may be present after a cataract surgery. In particular, such a correction is made by introducing regions of opacity into the material of the IOL that will affect the passage of light through the IOL, and thereby correct the vision defect. The regions of opacity that effectively establish optical barriers will vary in their orientation within the IOL depending on the particular defect that is to be corrected (e.g. “depth of focus”, or “dysphotopsia”). Also, the level of the opacity in regions of opacity can be varied.

As envisioned for the present invention, the IOL will be made of a substantially transparent material, such as silicon or a modified acrylic. With these materials, and others unspecified here, optical barriers can be created inside the IOL using laser techniques. Specifically, the optical barriers can be established either by creating microbubbles in the material of the IOL, or by converting inclusions that have been previously embedded in the material.

Structurally, the present invention involves a system for optically altering an Intraocular Lens (IOL), in situ. The system is essentially computer-controlled, and includes a laser unit for generating a laser beam and for focusing the laser beam to a focal spot. Additionally, the system includes a detector (imaging unit) for creating images of the IOL. In detail, the laser unit can be of any type known in the pertinent art that will generate a pulsed laser beam (e.g. a so-called femtosecond laser). And, the detector (imaging unit) can be selected from a group that includes an Optical Coherence Tomography (OCT) device, a Scheimpflug device, a two-photon imaging unit, or any other imaging (detector) device known in the pertinent art.

As impliedly noted above, both the laser unit and the detector (imaging unit) are connected to a computer. With this connection, the computer is able to receive images from the imaging unit, and to then use the images for control of the laser unit in guiding movements of the laser beam's focal spot. In accordance with a computer program, the focal spot is moved along a predetermined path inside the IOL, relative to an operational axis that is defined by the IOL. Preferably, this operational axis is identified as an optical axis of a patient. In any event, movements of the focal spot are accomplished to optically alter material of the IOL by forming an optical barrier of the altered material inside the IOL.

As envisioned for the present invention, there are essentially two particular orientations of interest for the creation of optical barriers inside an IOL. In one embodiment, the altered material forms an annulus that is centered on the operational axis. Typically, the annulus will be oriented in a plane substantially perpendicular to the axis, and it will have an inner radius “rᵢ”, and an outer radius “rₒ”. Further, it may be desirable for the annulus to have an increasing opacity gradient in the annulus in a direction from “rᵢ” to “rₒ”. This configuration for an optical barrier is particularly useful for increasing the depth of focus in the treatment of myopia. In another embodiment, the altered material is oriented to form a hollow cylindrical surface that is centered on the axis, and located at a distance “d” from the axis. This configuration for an optical barrier is particularly useful for minimizing the effects of dysphotopsia.

For the present invention, an optical alteration of the material in the IOL can be accomplished with the laser beam by creating microbubbles in the material. Alternatively, the material of the IOL can be pre-seeded with a plurality of inclusions (e.g. chromophores), and an optical alteration of material in the IOL is accomplished via an interaction of the plurality of inclusions with the laser beam that converts the plurality of inclusions into an opacity.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a diagram of the operational components of the present invention;

FIG. 2 is a perspective view of an Intraocular Lens (IOL) that includes an annular shaped optical barrier; and

FIG. 3 is a perspective view of an Intraocular Lens (IOL) that includes a cylindrical shaped optical barrier.

### DESCRIPTION OF THE PREFERRED EMBODIMENTS

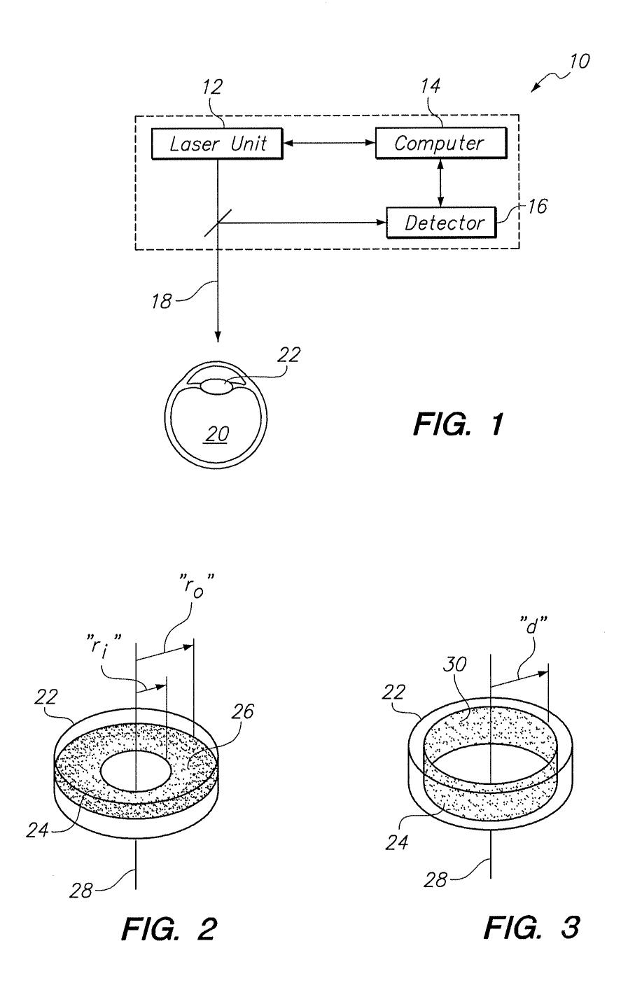

Referring initially to FIG. 1 a system in accordance with the present invention is shown, and is generally designated 10. As shown, the system 10 includes a laser unit 12 that is connected to a computer 14. Further, the computer 14 is connected to a detector 16. With the concerted cooperation of these components (i.e. laser unit 12, computer 14 and detector 16), a laser beam 18 is generated, focused and directed toward an eye 20. The laser beam 18 is then employed by the system 10 to alter the material of an Intraocular Lens (IOL) 22 for the purpose of correcting vision defects that may have been introduced by the IOL 22 after a cataract surgery.

For purposes of the present invention, the laser unit 12 will preferably be capable of generating a pulsed laser beam 18. Depending on the material used for the manufacture of the IOL 22 (e.g. silicon or a modified acrylic), the duration of pulses in the laser beam 18 can be varied (i.e. femtosecond, picosecond or nanosecond). Most likely, however, it will be a femtosecond pulsed laser beam 18. In addition to variable pulse durations, the pulse rate of the laser beam 18 can also be varied as required. The detector 16 will typically be an imaging unit of a type well known in the pertinent art that is capable of creating three-dimensional images inside the eye 20. For example, the detector 16 may be an Optical Coherence Tomography (OCT) device, a Scheimpflug device, or a two-photon imaging unit.

As envisioned for the present invention, the IOL 22 will be made of a transparent material, such as silicon or a modified acrylic. Accordingly, the IOL 22 is susceptible to photoalteration by the laser beam 18. More particularly, for purposes of the present invention, the laser beam 18 is employed to create microbubbles, or to convert inclusions. In either case, the result is a creation of a plurality of microbubbles/inclusions 24 as substantially shown in FIGS. 2 and 3. The consequence of the photoalteration will be to introduce an opacity in the material of the IOL 22 at the point where the focal spot of the laser beam 18 interacts either directly with the material of the IOL 22 (e.g. creation of microbubbles) or with inclusions (e.g. conversion of chromophores) that have been previously embedded into the material of the IOL 22.

In FIG. 2 a pattern of microbubbles/inclusions 24 is shown which creates an annular-shaped optical barrier 26 inside the IOL 22. As shown, the optical barrier 26 is centered on an axis 28 that is defined by the IOL 22, and it is oriented in a plane that is substantially perpendicular to the axis 28. Preferably, the axis 28 is coincident with an optical axis of the eye 20 in which the IOL 22 has been implanted. For the present invention, it is envisioned that the optical barrier 26 will be substantially flat, and it will extend outwardly from the axis 28 through a distance from an inner radial distance “rᵢ” to an outer radial distance “rₒ”.

It is also indicated in FIG. 2 that the density of microbubbles/inclusions 24 can be varied. In this case the optical barrier 26 is shown to have a gradient of increasing opacity in a direction from “rᵢ” to “rₒ”. As will be appreciated by the skilled artisan, this increasing opacity gradient can be established as a result of an increasing density of the microbubbles/inclusions 24.

Operationally, the optical barrier 26 shown in FIG. 2 is created with the system 10 by focusing the laser beam 18 to a focal spot at locations inside the IOL 22 where microbubbles/inclusions 24 are to be created. The laser beam 18 is then activated by the computer 14 to either create a microbubble or convert an inclusion at each location. This interaction between the laser beam 18 and the IOL 22 is then continued by moving the focal spot of the laser beam 18 over a predetermined area inside the IOL 22. As noted above, during such a movement of the laser beam 18, the density of the resultant microbubbles/inclusions 24 (and thus the opacity of the optical barrier 26) can be varied as desired.

As envisioned for the present invention, an operation of the system 10 is conducted in accordance with control by the computer 14. For such control purposes, the detector 16 is used to monitor the movement of the focal spot of laser beam 18, and to create real time images of the microbubbles/inclusions 24 as they are created to establish the optical barrier 26. The computer 14 can then compare these images with the intended, pre-programmed locations of the microbubbles/inclusions 24 for the optical barrier 26. The computer 14 can then use the comparisons for closed loop control of the laser unit 12 to establish the optical barrier 26.

FIG. 3 shows an optical barrier 30 that is established with microbubbles/inclusions 24 in a manner that is similar to that disclosed above for the optical barrier 26. In this case, however, as shown in FIG. 3 the optical barrier 30 is formed as a cylindrical surface that is centered on the axis 28, and is located at a radial distance “d” from the axis 28. As will be appreciated by the skilled artisan, the optical barrier 26 (see FIG. 2) is appropriate for increasing the depth of focus of eye 20, such as in the case of myopia. On the other hand, the optical barrier 30 (FIG. 3) is appropriate for the treatment of dysphotopsia. In both cases, the optical barriers 26 and 30 can be established by the laser system 10, in situ, inside the IOL 22, while the IOL 22 remains implanted in the eye 20.

While the particular System and Method for In Situ Creation of a Small Aperture Intraocular Lens as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for optically altering an Intraocular Lens (IOL), in situ, which comprises:
a laser unit for generating a laser beam and for focusing the laser beam to a focal spot;
an imaging unit for creating images of the IOL, wherein the IOL defines an operational axis; and
a detector connected to the laser unit and to the detector for using images received from the detector to guide movement of the laser beam focal spot, wherein the focal spot is moved within an area inside the IOL, relative to the axis, to optically alter material of the IOL to form an optical barrier of the altered material inside the IOL.

2. A system as recited in claim 1 wherein the altered material forms an annulus centered on the axis, with the annulus oriented in a plane substantially perpendicular to the axis, and wherein the annulus has an inner radius “rᵢ” measured from the axis, and an outer radius “rₒ” measured from the axis, and wherein the IOL is made of a substantially transparent material and the altered material introduces an opacity into the material of the IOL.

3. A system as recited in claim 2 wherein the annulus has an increasing opacity gradient in the annulus in a direction from “rᵢ” to “rₒ”.

4. A system as recited in claim 1 wherein the altered material forms a hollow cylindrical surface centered on the axis with the cylindrical surface located at a distance “d” from the axis, and wherein the IOL is made of a substantially transparent material and the altered material introduces an opacity into the material of the IOL.

5. A system as recited in claim 1 wherein an optical alteration of the material in the IOL is accomplished with the laser beam by creating microbubbles in the material.

6. A system as recited in claim 1 wherein the material of the IOL is pre-seeded with a plurality of inclusions, and an optical alteration of the material in the IOL is accomplished by a conversion of the plurality of inclusions in response to an interaction of the plurality of inclusions with the laser beam.

7. A system as recited in claim 6 wherein the inclusions are chromophores.

8. A system as recited in 1 wherein the IOL material is selected from a group which comprises silicon and a modified acrylic.

9. A system as recited in claim 1 wherein the laser unit generates a pulsed laser beam and the detector is selected from a group comprising an Optical Coherence Tomography (OCT) device, a Scheimpflug device, and a two-photon imaging unit.

10. A system as recited in claim 1 wherein the operational axis is identified as an optical axis of a patient.

11. A method for optically altering an Intraocular Lens (IOL), in situ, which comprises the steps of:
creating in situ images of the IOL;
identifying an operational axis for the IOL, wherein the operational axis is based on information from the images obtained during the creating step;
defining a predetermined area inside the IOL relative to the axis;
generating a pulsed femtosecond laser beam;
focusing the laser beam to a focal spot;
moving the laser beam focal spot along the predetermined path with reference to the images obtained during the creating step; and
optically altering material of the IOL during the moving step to form an optical barrier of the altered material inside the IOL.

12. A method as recited in claim 11 wherein the altered material forms an annulus centered on the axis with the annulus oriented in a plane substantially perpendicular to the axis, and wherein the annulus has an inner radius “rᵢ” measured from the axis, and an outer radius “rₒ” measured from the axis, and wherein the IOL is made of a substantially transparent material and the altered material introduces an opacity into the material of the IOL, and further wherein the annulus has an increasing opacity gradient in the annulus in a direction from “rᵢ” to “rₒ”.

13. A method as recited in claim 11 wherein the altered material forms a hollow cylindrical surface centered on the axis with the cylindrical surface located at a distance “d” from the axis, and wherein the IOL is made of a substantially transparent material and the altered material introduces an opacity into the material of the IOL.

14. A method as recited in claim 11 wherein an optical alteration of the material in the IOL is accomplished with the laser beam by creating microbubbles in the material.

15. A method as recited in claim 11 wherein the material of the IOL is pre-seeded with a plurality of inclusions, and an optical alteration of the material in the IOL is accomplished by a conversion of the plurality of inclusions in response to an interaction of the plurality of inclusions with the laser beam.

16. A computer program product for use with a computer to optically alter an Intraocular Lens (IOL), wherein the computer program product comprises program sections for respectively: creating in situ images of the IOL; receiving the in situ images of the IOL for use in identifying an operational axis for the IOL; defining a predetermined area inside the IOL relative to the operational axis; focusing a laser beam to a focal spot; and moving the laser beam focal spot within the predetermined area to optically alter material of the IOL to form an optical barrier of the altered material inside the IOL.

17. A computer program product as recited in claim 16 wherein the altered material forms an annulus centered on the axis with the annulus oriented in a plane substantially perpendicular to the axis, and wherein the annulus has an inner radius “rᵢ” measured from the axis, and an outer radius “rₒ” measured from the axis, and wherein the IOL is made of a substantially transparent material and the altered material introduces an opacity into the material of the 10L, and further wherein the annulus has an increasing opacity gradient in the annulus in a direction from “rᵢ” to “rₒ”.

18. A computer program product as recited in claim 16 wherein the altered material forms a hollow cylindrical surface centered on the axis with the cylindrical surface located at a distance “d” from the axis, and wherein the IOL is made of a substantially transparent material and the altered material introduces an opacity into the material of the IOL.

19. A computer program product as recited in claim 16 wherein an optical alteration of the material in the IOL is accomplished with the laser beam by creating microbubbles in the material.

20. A computer program product as recited in claim 16 wherein the material of the IOL is pre-seeded with a plurality of inclusions, and an optical alteration of the material in the IOL is accomplished by a conversion of the plurality of inclusions in response to an interaction of the plurality of inclusions with the laser beam.
